(19) United States
(12) Patent Application Publication
(10) Pub. No.: US 2022/0326209 A1
(43) Pub. Date: Oct. 13, 2022

(54) Paper-Based Microfluidic Don-Chip for Rapid and Low-Cost Deoxynivalenol Quantification in Foods, Feeds and Feed Ingredients

(71) Applicant: University of Manitoba, Wiinnipeg (CA)

(72) Inventors: Qian Jiang, Winnipeg (CA); Jiandong Wu, Winnipeg (CA); Francis Lin, Winnipeg (CA); Chengbo Yang, Winnipeg (CA)

(21) Appl. No.: 17/762,829

(22) PCT Filed: Sep. 28, 2020
(86) PCT No.: PCT/CA2020/051289

§ 371 Date: Mar. 23, 2022

Related U.S. Application Data
(60) Provisional application No. 62/906,441, filed on Sep. 26, 2019.

(51) Int. Cl. G01N 33/02; B01L 3/00; G01N 33/569

(52) U.S. Cl. CPC G01N 33/02; B01L 3/5023; G01N 33/56961; B01L 2300/069; B01L 2300/0825; B01L 2300/126; B01L 2300/161; B01L 2400/0406; G01N 2333/37

(57) ABSTRACT

A rapid, low-cost, portable and reliable method for on-site detection of deoxynivalenol (DON), a representative mycotoxin predominantly occurring in grains, would be helpful to control mycotoxin contamination. Herein, a paper-based microfluidic chip capable of measuring deoxynivalenol (DON-Chip) in foods, feeds and feed ingredients was developed. As discussed herein, the DON-Chip incorporated a colorimetric competitive immunoassay into a paper microfluidic device and used gold nanoparticles as a signal indicator. Furthermore, a novel ratiometric analysis method was used to improve signal resolvability at low concentrations of DON. Detection of DON in aqueous extracts from solid foods, feeds or feed ingredients was successfully validated with a detection range from 0.01-20 ppm (using dilution factors from 10-10⁴). Compared with conventional methods, the novel DON-Chip greatly reduces the cost and time of mycotoxin detection in the food and feed industry.

### PRIOR APPLICATION INFORMATION

The instant application claims the benefit of U.S. Provisional Patent Application 62/906,441, filed Sep. 26, 2019 and entitled “A paper-based microfluidic DON-Chip for rapid and low-cost deoxynivalenol quantification in foods, feeds and feed ingredients”, the entire contents of which are incorporated herein by reference for all purposes.

### BACKGROUND OF THE INVENTION

Mycotoxins are toxic chemicals produced by fungi that infect crops. It is reported that typically more than 25% of the harvested crops have been contaminated with mycotoxin (1). More than 200 species of trichothecene, which are divided into four groups, have been found so far (2). Vomitoxin is one of the trichothecene mycotoxin produced by Fusarium (3). The main compounds of vomitoxin consist of deoxynivalenol (DON), 3-acetyl deoxynivalenol, and 15-acetyl deoxynivalenol, which are widely present in cereals such as wheat, barley, and corn (4). Vomitoxin contaminations pose threats to the health of humans and animals, especially to immune functions (5-7). Specifically, the ingestion of foods contaminated with vomitoxins can cause immunosuppression or immune overstimulation, resulting in some acute poisoning symptoms such as anorexia (8), vomiting (9), diarrhea (10), fever, and unresponsiveness (11). In severe cases, vomitoxin could damage the hematopoietic system and cause death (12). Due to the toxic effects of vomitoxins on the health of humans and animals, there are currently 37 countries in the world that have relevant limits for vomitoxins in foods or feeds. The USFDA stipulates that the safety standard for vomitoxins in foods is 1 ppm (13). The safety standards for vomitoxins in the feeds are animal species-dependent, for example, it is lower than 1 ppm for swine and 5 ppm for ruminant and poultry (14). Vomitoxin is represented in more than 90% of all mycotoxin-contaminated samples, and its presence usually indicates that other mycotoxins are also present (15). The laboratory detection methods for vomitoxin mainly consist of high-performance liquid chromatography (HPLC), enzyme-linked immunosorbent assay (ELISA), and liquid chromatography-tandem mass spectrometry (LC-MS), whose pre-processing steps are cumbersome and analysis time is long. The high cost and low efficiency of these laboratory methods makes them difficult to be widely used in food or feed quality supervision and control.

Rapid, sensitive and accurate methods for vomitoxin quantification in a non-laboratory environment are essential for toxicological analysis and risk assessment of foods or feed products. At present, the lateral flow immunoassay (LFIA), also known as immunochromatographic assay (ICA) or strip test, is currently used for qualitative, semi-quantitative, or quantitative monitoring of vomitoxin in a non-laboratory environment. Sandwich and competitive targeting methods could be used for immunoassays. Sandwich immunoassays are commonly used to measure large analytes with multiple epitopes, while competitive immunoassays are the choice for quantification of small analytes which have low molecular weight or only a single specific epitope (16). As vomitoxin is a small molecule and does not exhibit more than one epitope, the competitive method is used for qualitative and quantitative detection of vomitoxins. Specifically, an indicator (labeled with vomitoxin or vomitoxin antibody) reacts with capturing molecules (vomitoxin antibody or vomitoxin) deposited in the test area (17-19). Vomitoxin in the sample competes for the binding sites with the capturing molecules (vomitoxin antibody or vomitoxin) on the test area, leading to non-aggregation of indicators in the test area.

Due to heterogeneous distribution of vomitoxins in the same batch of food or feed products, density-based sampling and replicate detections are needed for assessment of mycotoxin contamination, which increases the detection cost (20). Many commercial immunocolloidal gold rapid detection kits have been developed for detecting vomitoxins in foods or feed products. However, these commercial kits are relatively high priced and not sensitive for on-site vomitoxin detection. Microfluidic analytical devices have been considered as a promising alternative to the traditional tests. Various materials can be used for fabricating microfluidic devices such as polymers, thermoplastic, glass, cloth, and paper (21). Among them, the microfluidic paper based analytical device (μPAD) offers the benefits of low-cost, easy fabrication, and self-powered fluidic transport by capillary force (22). The μPAD can control fluidic transport within hydrophilic channels defined by hydrophobic barriers (23). Recently, μPAD has been increasingly used for various chemical, biochemical, and biological applications (24-26).

In the present study, we developed a μPAD-based immunoassay for rapid and low-cost detection of DON, called DON-Chip. We chose DON for this initial assay because this vomitoxin is present in more than 90% of all mycotoxin-contaminated samples, and its presence is usually a good indicator that other mycotoxins are also present. The paper-based microfluidic immunoassay was realized by competitive immunoreaction leveraged gold nanoparticle-based colorimetric signals. These signals were captured using a portable USB microscope. The developed DON-Chip was successfully validated by DON standards and different food, feed and feed ingredient samples. The useful features of this DON-Chip are fast testing (within 12 min), low-cost (<2 US dollars of material cost per test), high reproducibility, and integration with a portable imaging system for easy signal readout. Overall, the DON-Chip provides an excellent example of microfluidic paper-based technology for rapid and low-cost detection of mycotoxins in foods and feed products.

### SUMMARY OF THE INVENTION

According to a first aspect of the invention, there is provided a method for detecting levels of deoxynivalenol in a sample comprising:

providing an assay support comprising a sample loading area connected by a channel to a first test area and a second test area;

said sample loading area comprising a quantity of anti-deoxynivalenol compound binding antibodies;

said first test area comprising a quantity of deoxynivalenol compound bound to a carrier;

said second test area comprising a quantity of anti-deoxynivalenol compound binding antibodies binding reagent;

wherein said sample flows from the sample loading area along the channel to the first test area and then along the channel to the second test area;

loading a sample to be tested for a deoxynivalenol compound onto the sample loading area such that contents of the sample interact with the quantity of anti-deoxynivalenol compound binding antibodies, a portion of said quantity of anti-deoxynivalenol compound binding antibodies forming anti-deoxynivalenol compound binding antibody:deoxynivalenol compound complexes, a remaining portion of the quantity of anti-deoxynivalenol compound binding antibodies remaining unbound anti-deoxynivalenol binding antibodies;

said sample comprising the anti-deoxynivalenol compound binding antibody:deoxynivalenol complexes and the unbound anti-deoxynivalenol compound binding antibodies flowing along the channel to the first test area, said unbound anti-deoxynivalenol compound binding antibodies binding to the quantity of deoxynivalenol compound bound to a carrier and being retained in the first test area;

said sample comprising the anti-deoxynivalenol compound binding antibody:deoxynivalenol compound complexes continuing to flow along the channel to the second test area, said anti-deoxynivalenol compound binding antibody:deoxynivalenol compound complexes binding to the quantity of anti-deoxynivalenol compound binding antibodies binding reagent and being retained in the second test area; and

measuring the deoxynivalenol compound level in the sample by detecting the anti-deoxynivalenol compound binding antibodies at the first test area and/or detecting the anti-deoxynivalenol compound binding antibodies at the second testing area.

According to another aspect of the invention, there is provided a method for manufacturing a device for detecting deoxynivalenol in a sample comprising:

providing an assay support comprising a sample loading area connected by a channel to a first test area and a second test area;

depositing a quantity of anti-deoxynivalenol compound binding antibodies at the sample loading area;

depositing a quantity of anti-deoxynivalenol compound bound to a carrier at the first test area; and

depositing a quantity of anti-deoxynivalenol compound binding antibody binding reagent at the second testing area.

### BRIEF DESCRIPTION OF THE DRAWINGS

FIG. 1. Schematic of DON-Chip fabrication and brief measurement procedures. DON, deoxynivalenol; DON-BSA, the deoxynivalenol conjugated bovine serum albumin.

FIG. 2. Illustration of the DON measurement principle using the DON-Chip. DON, deoxynivalenol; AuNPs, gold-nanoparticles; DON-BSA, deoxynivalenol conjugated bovine serum albumin.

FIG. 3. (A) Representative images of T1 and T2 areas in the DON-Chips using different concentrations of DON standard. (B) A calibration curve using T1 signals in the DON-Chip. (C) A calibration curve using T2 signals in the DON-Chip. (D) A calibration curve using T1/T2 in the DON-Chip. Two chips (total of four channels) were used for each concentration.

FIG. 4. The linear fit between the deoxynivalenol (DON) results obtained from enzyme-linked immunosorbent assay (ELISA) and DON-Chip methods (R²=0.9991; slope=0.8988). Twenty food, feed and feed ingredient samples were used in the DON measurements. Mean values of each detection (4 replications for each sample) were used for the linear fit. The Y-axis and the X-axis represent the values obtained from the DON-Chip method and the ELISA kit,

FIG. 5. Optimization of figurate designs for DON-Chip. The capillary speed of the chips with different figurate designs was tested. The channels in the original designs of the Chip are 1.0 mm width (Original 1), 2.0 mm width (Original 2). The channels in the optimized DON-Chip are 1.5 mm width, and circular arc designed. After blocking these Chips with 0.2% BSA, 20 μL of PBS was loaded in the loading areas of these Chips, and the images shown were captured after 60 seconds capillary flowing.

FIG. 6. Optimization of DON-BSA concentration in the T1 area. 0.1 μL of 0.475, 0.950, 1.900 and 3.800 μg/μL DON-BSA were deposited in the T1 area of 4 DON-chips, respectively. The T2 areas were deposited with 1.0 μg/μL of anti-mouse IgG. During the detection, 20 μL of PBS (pH=7.2) was loaded in the conjugate pad of these DON-chips. DON-BSA, deoxynivalenol conjugated bovine serum albumin.

FIG. 7. Total signaling intensity (T1₊ T2) in the channels are shown. For the detections, the DON-Chips are loaded with 20 μL DON standards at concentrations of 0.0, 1.0, 2.0, 4.0, 8.0, 16.0, and 20.0 ng/mL.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Mycotoxin contamination causes over 5 billion dollars of economic loss per year in the North American food and feed industry. A rapid, low-cost, portable and reliable method for on-site detection of deoxynivalenol (DON), a representative mycotoxin predominantly occurring in grains, would be helpful to control mycotoxin contamination. Herein, a paper-based microfluidic chip capable of measuring deoxynivalenol (DON-Chip) in foods, feeds and feed ingredients was developed. As discussed herein, the DON-Chip incorporated a colorimetric competitive immunoassay into a paper microfluidic device and used gold nanoparticles as a signal indicator. Furthermore, a novel ratiometric analysis method was used to improve signal resolvability at low concentrations of DON. Detection of DON in aqueous extracts from solid foods, feeds or feed ingredients was successfully validated with a detection range from 0.01-20 ppm (using dilution factors from 10-10⁴). Compared with conventional methods, the novel DON-Chip greatly reduces the cost and time of mycotoxin detection in the food and feed industry.

As discussed herein, with careful optimizations in device design, reagent concentration and reaction conditions, the DON-Chip can be used for on-site measurement of DON concentration in real-world foods, feeds and feed ingredients within 12 min, with a detection range from 0.01-20 ppm. It is worth noting that the present DON-Chip is the first competitive immunoassay that combines the complementary signals of low and high DON concentration samples for a more complete and realistic measurement. Moreover, the ratiometric value between these two signals provide lower LODs and better resolvability at low concentrations. Overall, the DON-Chip offers a low-cost portable alternative for mycotoxin detection with strong implications for improving animal health and food safety.

According to an aspect of the invention, there is provided a method for detecting levels of deoxynivalenol in a sample comprising:

providing an assay support comprising a sample loading area connected by a channel to a first test area and a second test area;

said sample loading area comprising a quantity of anti-deoxynivalenol compound binding antibodies;

said first test area comprising a quantity of deoxynivalenol compound bound to a carrier;

said second test area comprising a quantity of anti-deoxynivalenol compound binding antibodies binding reagent;

wherein said sample flows from the sample loading area along the channel to the first test area and then along the channel to the second test area;

loading a sample to be tested for a deoxynivalenol compound onto the sample loading area such that contents of the sample interact with the quantity of anti-deoxynivalenol compound binding antibodies, a portion of said quantity of anti-deoxynivalenol compound binding antibodies forming anti-deoxynivalenol compound binding antibody:deoxynivalenol compound complexes, a remaining portion of the quantity of anti-deoxynivalenol compound binding antibodies remaining unbound anti-deoxynivalenol binding antibodies;

said sample comprising the anti-deoxynivalenol compound binding antibody:deoxynivalenol complexes and the unbound anti-deoxynivalenol compound binding antibodies flowing along the channel to the first test area, said unbound anti-deoxynivalenol compound binding antibodies binding to the quantity of deoxynivalenol compound bound to a carrier and being retained in the first test area;

said sample comprising the anti-deoxynivalenol compound binding antibody:deoxynivalenol compound complexes continuing to flow along the channel to the second test area, said anti-deoxynivalenol compound binding antibody:deoxynivalenol compound complexes binding to the quantity of anti-deoxynivalenol compound binding antibodies binding reagent and being retained in the second test area; and

measuring the deoxynivalenol compound level in the sample by detecting the anti-deoxynivalenol compound binding antibodies at the first test area and/or detecting the anti-deoxynivalenol compound binding antibodies at the second testing area.

In some embodiments of the invention, the anti-deoxynivalenol compound antibodies comprise a detectable label.

It is of note that any suitable detectable label known in the art for use with antibodies may be used within the invention. For example, the detectable label may be a gold-nanoparticle or a fluorescent microsphere.

In some embodiments of the invention, the anti-deoxynivalenol compound antibodies are labeled with gold nanoparticles.

As discussed herein, in these embodiments, the test is a colorimetric test. Accordingly, a wide variety of detection methods may be used, including for example a cell phone's camera.

In some embodiments, the assay support is a paper-based microfluidic chip.

For example, the paper-based microfluidic chip may be composed of nitrocellulose paper.

As will be appreciated by one of skill in the art, compared with the traditional well-plate-based assay, the use of a paper microfluidic device has several advantages: a) it has a faster reaction time. The nitrocellulose paper consists of large numbers of micropores (0.45 μm) that favor the contacts and reactions between antibody and antigens (all the reactions can be done in 10s, during the liquid flow). In a well plate-based ELISA assay, the molecules are all in Brownian motion within a microplate, and it takes a longer time (40 min in 37° C.) to finish the reaction. b) The reagents are preloaded in the chip thus the chip is self-sufficient for the assay, as discussed herein. c) Easy operation, as capillary force is used to drive the flow. d) Compared with the traditional lateral flow assay, the paper microfluidic device not only consumes fewer reagents and samples but one assay support could be easily developed to achieve multiplex detection, that is, to process multiple samples.

The sample may be a food sample or a feed sample. As will be appreciated by one of skill in the art, an ingredient of a food product or feed product would also be considered a food sample or feed sample.

As discussed herein, in some embodiments, the deoxynivalenol compound level is determined by detecting the anti-deoxynivalenol compound binding antibodies at the first test area and detecting the anti-deoxynivalenol compound binding antibodies at the second testing area.

As discussed below, because the total amount of anti-deoxynivalenol compound binding antibodies in the assay support is known, detecting the amount in either one test area or both can be used as a measurement of the level of the deoxynivalenol compound in the sample for example by using previously prepared calibration curves as discussed herein. It is of note that the calibration curves do not necessarily need to be repeated for each test.

As discussed herein, in some embodiments, the deoxynivalenol compound level is determined by the ratio of anti-deoxynivalenol compound binding antibodies at the first test area to the anti-deoxynivalenol compound binding antibodies at the second testing area.

As discussed herein, in some embodiments, the width of the channel may be from 1.0-2.0 mm and the length of the channel may be from 8 mm-16 mm.

As will be appreciated by one of skill in the art, the dimensions of the chip are closely associated with detection speed and cost. A chip with larger dimensions would consume more antibodies to get a proper signal intensity, while a smaller dimension limits the liquid flow speed which may be acceptable under certain circumstances.

In some embodiments of the invention, the channel has curved corners which achieves the best flow speed among the designs tested. As per above, in some embodiments, other arrangements are acceptable in certain circumstances.

The first testing area and the second testing area may be separated by a separation zone. As will be appreciated by one of skill in the art, without a separation between the two test areas, the reagents bound in the T1 area and T2 area may cross-contaminate and/or it may not be easy to distinguish the boundary of the distinct signals emitting from these two areas. In some embodiments, the separated area or separation zone has a different width, that is, has a larger width than the channel. For example, the separation zone may be 2 mm wide while the channel width may be 1.5 mm.

The “deoxynivalenol compound” as used herein refers to a compound selected from the group consisting of deoxynivalenol, 3-acetyl deoxynivalenol and 15-acetyl deoxynivalenol.

In some embodiments, the assay support further comprises an absorbent zone and the sample flows along the channel from the second test area to the absorbent zone. As will be appreciated by one of skill in the art, the absorbent zone at one end of the channel promotes flow from the sample loading area, past the first test area and the second test area.

The anti-deoxynivalenol compound binding antibodies binding reagents may be any suitable agent known in the art that will specifically bind the anti-deoxynivalenol compound binding antibodies. In some embodiments, these reagents are secondary antibodies. As is known to those of skill in the art, secondary antibodies are used in the art to bind to the primary antibody to assist in detection, sorting and purification of target antigens. To enable detection, the secondary antibody must have specificity for the antibody species and isotype of the primary antibody being used.

According to another aspect of the invention, there is provided a method for manufacturing a device for detecting deoxynivalenol in a sample comprising:

providing an assay support comprising a sample loading area connected by a channel to a first test area and a second test area;

depositing a quantity of anti-deoxynivalenol compound binding antibodies at the sample loading area;

depositing a quantity of anti-deoxynivalenol compound bound to a carrier at the first test area; and

depositing a quantity of anti-deoxynivalenol compound binding antibody binding reagent at the second testing area.

As discussed herein, within an ideal detecting environment (room temperature=24° C., pH value=6-8), all the parameters (T1, T2, T1/T2, and T2/T1) are accurate indicators for DON quantitation. According to our analysis from the raw data and these calibration curves, the ratiometric parameters (T1/T2, T2/T1) could achieve lower LODs (T1/T2 of 0.432 ng/mL vs T1 of 0.644 ng/mL), higher recovery ratio and higher resolution in the marginal concentration ranges.

For example, the immunoreactions both in T1 area and T2 area tend to be weaker in the morning due to a relatively low temperature, and it is expected that the signal intensity of both T1 and T2 will decrease by a similar percentage (For example, the T1 goes to be m*T1, and T2 goes to be n*T2, m, n<1). In this scenario, the ratiometric parameters (mT1/nT2, nT2/mT1) have a self-correcting capability by counteracting the errors from incomplete immunoreaction. The result from the values of mT1/nT2 and nT2/mT1 have better accuracy when compared with that from the sole signal values (m*T1 or n*T2).

Optimization of the DON-Chip. Speed, cost, reliability, and sensitivity are the most important parameters for an on-site detection system. We first optimized the DON-Chip to make a balance between these parameters through the following steps: 1) we chose an optimal channel dimension and pattern to achieve a fast speed for the sample flow; 2) we ensured an adequate amount of DON-BSA in the T1 area and secondary antibody in the T2 area, aiming to capture all the antibody-conjugated AuNPs; 3) we optimized the concentration of DON-BSA to ensure high sensitivity while reducing the cost; 4) we proposed an analysis method which combines the signals in T1 area and T2 area to generate the calibration curve and determine the DON concentration. The details of some optimizations are described as follows:

The effects of the channel dimension and pattern on the capillary flow speed are shown in FIG. 5. We tested channels with different widths of signal area, including 1.0 mm, 1.5 mm, and 2.0 mm. We verified that the channel with a 1.5 mm width and curved corners worked well for this application. After blocking the non-specific binding sites with 0.2% BSA, 20 μL of PBS was loaded in the loading area. The PBS in the optimized chip can quickly flow through the channels to the absorbent area within 60 seconds. In addition, a separating area (2.0 mm×2.0 mm) was placed between the T1 and T2 areas to avoid signal interference.

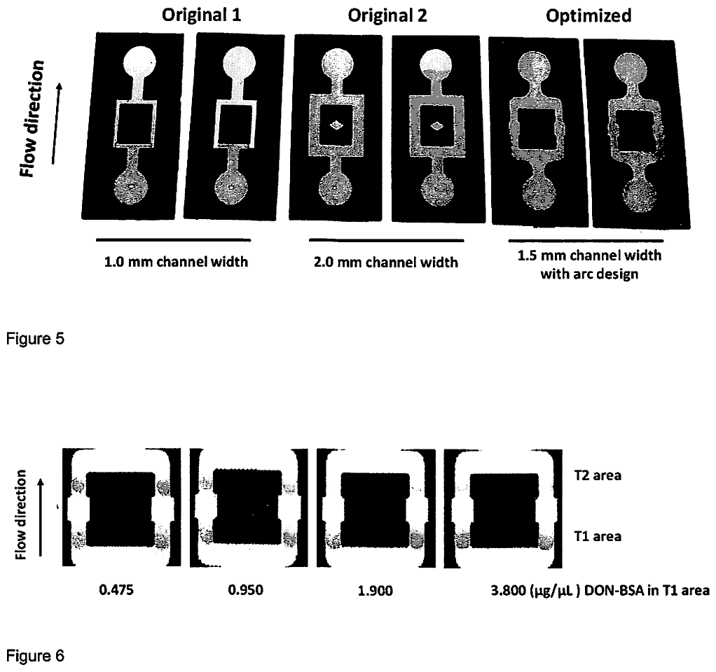

To optimize the concentration of DON-BSA, we deposited different concentrations of DON-BSA in the T1 area (ranging from 0 to 3.8 μg/μL) and assessed the signal after adding 2 uL of Anti-DON-AuNPs on the conjugate pad. As shown in FIG. 6, the signal intensity of T1 area increased with the increase of DON-BSA concentration and reached a plateau at 1.9 μg/μL. Meanwhile, the signal intensity in T2 area showed an opposite tendency when compared with that of the T1 area. Thus, we chose 1.9 μg/μL as the optimal concentration of DON-BSA.

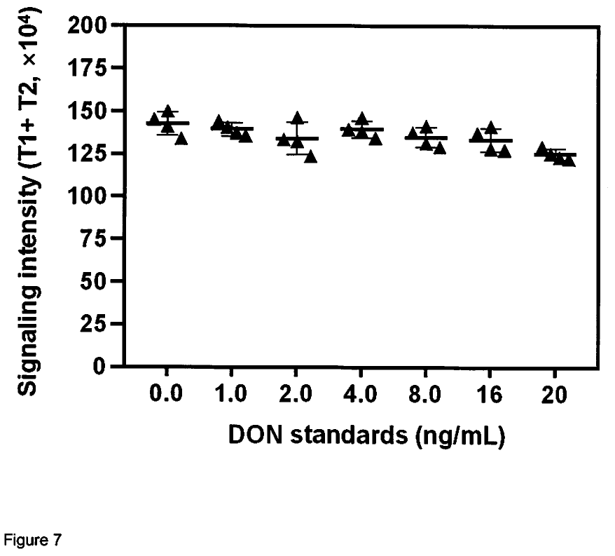

In each test, the volume of gold nanoparticles deposited in the DON-Chip was kept the same. Theoretically, the sum of the signals in T1 and T2 areas in one channel should be constant if all the antibody-conjugated gold nanoparticles were captured. We tested this hypothesis by summing the signals in T1 and T2 areas (T1+T2) in the channels after applying different concentrations of DON standards (0.0, 1.0, 2.0, 4.0, 8.0, 16.0, and 20.0 ng/mL). As shown in FIG. 7, the total signal (T1+T2) for different concentrations of DON are quite similar (CV=5.6%, P value=0.321 under one-way ANOVA test). Inappropriate storage and extreme detection conditions could damage the loaded reagents and adversely affect the reaction in the DON-Chip, leading to a decreased total signal (T1+ T2). Therefore, we further proposed that the total signal (T1+ T2) could be used as an effectiveness indicator for the test in the DON-Chip. A total intensity of 120×10⁴ was suggested as an effective cutoff for this purpose.

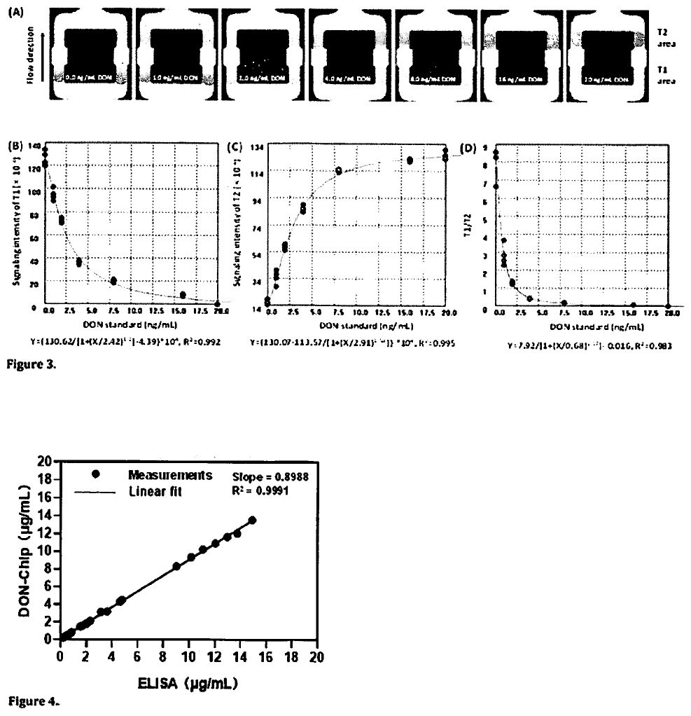

Calibration of the DON-Chip. As discussed herein, in some embodiments, the saturated binding concentration of DON in this DON-Chip is nearby 20 ng/mL. In the present study, we used concentrations of 0.0, 1.0, 2.0, 4.0, 8.0, 16, 20 ng/mL as DON standards to determine calibration curves. The representative images of signals in T1 and T2 in the DON-Chip are shown in FIG. 3A. The results clearly showed that DON concentration is negatively correlated to T1 signal and positively correlated to T2 signal. Thus, both T1 and T2 signals could be used to construct calibration values for DON detection. Furthermore, as mentioned above, the signals in T1 and T2 are negatively correlated and their sum keeps constant. Based on this finding, T1/T2 can be used as an additional ratiometric signal to determine DON concentration. This ratiometric signal would amplify the signal differences in low concentrations and would be helpful to improve the resolvability in this area. To the best of our knowledge, the present DON-chip is the first immunoreaction example combining the signals of antigen-deposited area (T1 area) and secondary antibody deposited area (T2 area) to indicate the final concentrations of antigens in the samples.

We then compared the performances of these three calibration curves for calculating the DON concentration in actual samples. The calibration curves using values of the T1, T2 and T1/T2 are shown in FIG. 3B-D. The logistic correlation coefficients (R²) were all above 0.98, indicating good correlations between the DON standard concentrations and the three signal parameters. From each calibration curve, we calculated the LOD based on the average value of blank signal values ±three times of the standard deviation. The calculated LODs are 0.644 ng/mL for T1 calibration curve, 0.468 ng/mL for T2 calibration curve, and 0.435 ng/mL for T1/T2 calibration curve. In order to further assess the performance of these three curves for determining different ranges of DON concentrations, we analyzed the recovery ratio and intra-CV of the detection data sets detecting the DON standards (1.0, 5.0, 15 ng/mL). As shown in Table 5, the data sets calculated by T1/T2 calibration curve has the best recovery ratio and a middle intra-CV among these three equations when detecting the 1.0 ng/mL DON standards. However, we observed the highest intra-CV among the data sets calculated by T1/T2 calibration curve when detecting the 5.0 and 15 ng/mL DON standards. The T2 calibration curve has higher R² (0.995 versus 0.983) and similar LOD (0.468 ng/mL versus 0.435 ng/mL) when compared with that of T1/T2. In the naturally contaminated grain samples, because the critical cut-off concentrations are usually located at the higher concentration areas of the calibration curves, where the T2 curve shows the highest steepness, we decided to use the T2 calibration curve to determine the DON concentration in these samples. On the other hand, as shown in Table 6, T1/T2 based calibration curve in low DON concentration range (0.5-1.0 ng/mL) has the highest resolvability. Therefore, in some embodiments, the parameter T1/T2 might be the best choice for determining if a sample is slightly contaminated with low concentrations of DON.

The USFDA stipulates that the safety standard for DON in foods is 1 ppm (13). The safety standards for DON in feeds are animal species-dependent, that is, lower than 1 ppm for swine and 5 ppm for ruminant and poultry (14). As will be appreciated by one of skill in the art, by applying different dilution factors from 10-1000, the detection range of this DON-Chip could be 0.01-20.0 ppm. The calculated LODs in the DON-Chip for the solid samples are 6.44 ppb, 4.68 ppb, and 4.35 ppb (dilute factor, 10) from the calibration curves of T1, T2, and T1/T2, respectively. As can be seen, the LODs for the DON-Chip are much lower than the LOD for a commercial ELISA kit (Table 7) and the safety standard for foods or feeds. These results indicate that DON-Chip has enough sensitivity and detection range to meet the safety regulations in food, feed and feed ingredient samples.

Stability of DON-Chip under different conditions. Considering that different pH values and many other species of mycotoxins occur in food, feed and feed ingredient samples, we tested the interferences of pH and ZEN, a frequent mycotoxin contaminant in cereals (27) on DON measurement using the DON-Chip. As shown in Table 1, adding ZEN (ranging from 1-15 ng/mL) in the standard samples did not affect the DON measurements.

As shown in Table 2, use of a dilution buffer having a pH ranging from 6.0-9.0 did not affect the DON measurements in the DON-Chip. However, the DON-Chip does not work in extreme acid (pH=3) or alkaline (pH=10) conditions. It is surmised that the ineffectiveness of these detections under extreme pH might be attributed to the denaturation of DON-antibody, and disassembly of anti-DON-AuNPs. It is of note that extreme pH conditions can be avoided by diluting the extracted supernatant into for example a PBS buffer before sample loading.

Efficacy of DON-Chip in spiked corn samples. To simulate DON detection in actual food or feed samples, we artificially added the DON standards to uncontaminated corn samples, and then extracted the DON from the spiked corn samples according to sample preparation and analytical procedures described in the methods section. As shown in Table 3, the recoveries of DON were in the range of 90-105%. All the CV values were lower than 10%, indicating good repeatability of this DON-Chip.

Applications of DON-Chip in food, feed and feed ingredient samples. To evaluate the capability of this DON-Chip in determining DON concentrations in real-world samples, the DON concentrations in 21 food, feed and feed ingredient samples were measured using the DON-Chip and a commercial DON ELISA kit. The samples were prepared using the same method described in the sample preparation section. A dilution factor of 100 was applied for the ELISA detections. A dilution factor of 1000 was applied for DDGS samples, 500 for corn, feed and unhusked rice samples, and 100 for wheat samples in DON-Chip detection. As shown in Table 4, the detection results from ELISA and DON-Chip for the same sample are quite similar (variation <15% between two methods). Only one data point from the DDGS sample (4) showed a significant difference (P<0.05) between these two methods.

The linear fit comparing the DON measurements obtained from ELISA and DON-Chip is shown in FIG. 4. The linear slope and correlation coefficient (R²) of the DON results are 0.8988 and 0.9991, respectively. Based on these results, the DON-Chip was validated as a reliable method for detecting DON in foods, feeds and feed ingredients.

Different DONs (deoxynivalenol, 3-acetyl deoxynivalenol, and 15-acetyl deoxynivalenol) have similar chemical structures, which presents a challenge to distinguish them using conventional immunoassays. However, the DON-Chip could be designed to leverage antibody cross-reactivity, such that one antibody capable of cross-reacting with all three common DONs in a sample could be detected to maximize efficiency and minimize overall cost. According to the data provided by the antibody provider, the values of cross reactivity between the DON-antibody to the deoxynivalenol, 3-acetyl deoxynivalenol, and 15-acetyl deoxynivalenol are 100%, 95% and 46%, respectively. Based on these values, all the three common DONs in the samples could be detected in the DON-Chip. Thus, the developed DON-Chip can be used as an effective tool to evaluate the total concentration of common DONs in commercial food and feed samples.

Comparison of DON-Chip to other methods. Table 7 shows the comparison of the DON-Chip with the ELISA kit and commercial strip in terms of LOD, assay time, detection range, cost, and on-site detection capability. The assay time of the DON-Chip is comparable with the commercial strip and both methods can be applied for on-site detection. Importantly, DON-Chip shows lowest LOD and cost. Therefore, the DON-Chip shows promise for on-site mycotoxin detection in the food and feed industry.

The invention will now be further explained and/or elucidated by way of examples; however, the invention is not necessarily limited to or by the examples.

### Example 1—Experimental Section

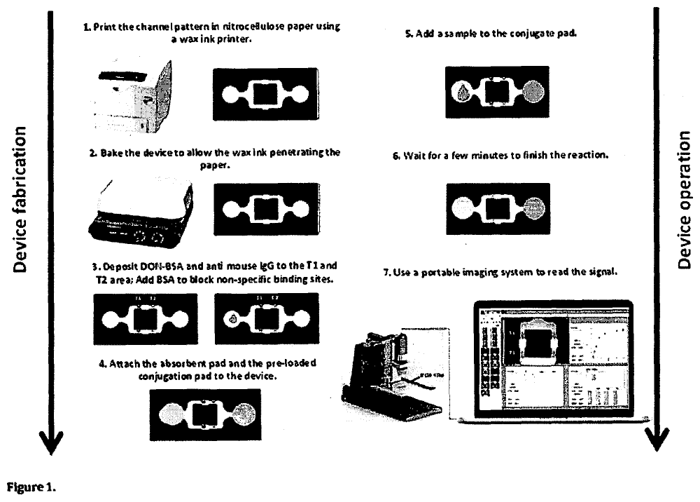

Materials. Purified deoxynivalenol standard (RN51481-10-8, >98%) was purchased from Sigma-Aldrich (Saint Louis, Mo., USA). BSA conjugated deoxynivalenol and deoxynivalenol polyclonal antibody (cross reactivities 100% to deoxynivalenol, 95% to 3-acetyl deoxynivalenol, and 46% to 15-acetyl deoxynivalenol) were purchased from Unibiotest Co., Ltd (Wuhan, Hubei, China). The GOLD nanoparticle conjugation Kit (ab188215) was purchasedfrom Abcam (Cambridge, Mass., USA). Goat anti-mouse IgG (40121) was purchased from Alpha Diagnostic Intl. Inc (San Antonio, Tex., USA). Nitrocellulose paper (HF09004X SS), absorbent paper FIG. 1 (CFSP 223000) and glass fiber membrane (CFDX 203000) were purchased from Millipore (Billerica, Mass., USA). The commercial DON ELISA Kit was purchased from Elabscience Biotechnology (Houston, Tex., USA).

Antibody-conjugated gold nanoparticles preparation. We conjugated the anti-DON antibody to the gold nanoparticles (AuNPs) according to the product manual and then purified the conjugations to remove the unconjugated antibody. Firstly, all the reagents were warmed to room temperature. The anti-DON antibody was diluted into the diluent buffer to a final concertation of 0.25 mg/mL. 120 μL of diluted anti-DON antibody was mixed with 420 μL reaction buffer thoroughly. 450 μL of the mixture was transferred to the AuNP vial and reconstituted the freeze-dried mixture by gently pipetting up and down for 20 min. Afterwards, 50 μL of quencher reagent was added to the vial and mixed gently. This process produced 500 μL of anti-DON antibody conjugated AuNPs (Anti-DON-AuNPs).

To remove the excess antibody, we washed the Anti-DON-AuNPs twice by 10 times volume of washing buffer and centrifuged the solution in a microfuge at 9,000 g for 30 minutes. The pellet was resuspended using 1:10 diluted quencher reagent and stored at 4° C. until use.

Paper-based device fabrication. As illustrated in FIG. 1, the pattern of the paper devices was designed using AutoCAD software (San Rafael, Calif., USA) and printed on nitrocellulose paper using a solid wax ink printer (ColorQube 8570, Xerox, Norwalk, Conn., USA). The paper was then heated on a hotplate at 125° C. for 25 seconds to let the melted wax ink penetrate the paper and form hydrophobic boundaries. Round 7 mm-diameter absorbent and conjugate pads were made using a puncher.

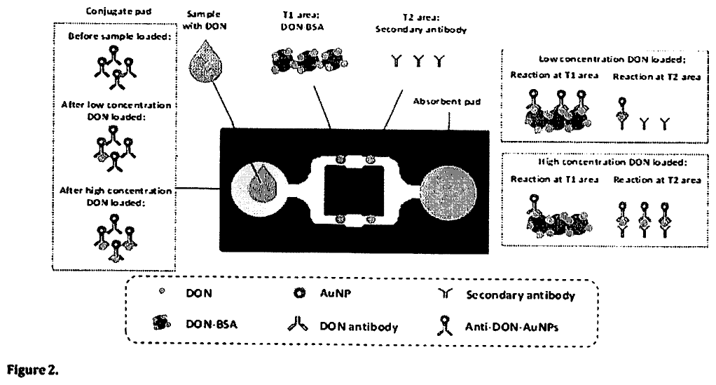

DON-BSA (0.1 μL at 1.9 μg/μL) was spotted at the test 1 (T1) area, and goat anti-mouse IgG (0.1 μL at 1.0 μg/μL) was deposited at the test 2 (T2) area. The spotted paper was dried overnight at room temperature. The dried paper was then blocked with 0.2% BSA in PBS for 20 minutes. A 2 μL working solution of Anti-DON-AuNPs was added to each conjugate pad. Conjugate pads were dried at room temperature and kept in 4° C. before use. The fully assembled DON-Chip consisted of the conjugate and absorbent pads attached to the patterned nitrocellulose paper as shown in FIG. 2. Assembled DON-Chips were used for experiments immediately or stored at 4° C. before use.

Sample preparation. Twenty-one naturally contaminated grain samples [distillers dried grains with soluble (DDGS), wheat, corn, feeds, and unhusked rice] were kindly provided by the Wallenstein Feed & Supply Ltd. (Wallenstein, ON, Canada). The samples were ground using a coffee grinder before mycotoxin extraction. One gram of each powder sample and 10.0 mL of water were added into a 50 mL Eppendorf tube and then mixed by a digital vortex mixer at 3000 times/min for 5 min. The solution was then centrifuged at 4,500 rpm for 5 min at 4° C., and 1 mL of the supernatant was transferred to an Eppendorf microcentrifuge tube for further analysis.

DON measurement using the DON-Chip. The operation and detection principles of the DON-Chip are illustrated in FIGS. 1 and 2, respectively. For detection, 2 μL of each sample was diluted into 18 μL PBS (loading buffer) and then dispensed onto the conjugate pad. Anti-DON antibody labeled with AuNP in the conjugate pad flowed along with the sample on the nitrocellulose paper via capillary action. The DON-Chip measurement was based on a competitive immunoassay. Samples with low concentrations of DON cannot bind all the Anti-DON-AuNPs, and the DON-BSAimmobilized at the T1 area will capture the unbound Anti-DON-AuNPs and show a color signal. Conversely, abundant DON in the sample binds specifically to the Anti-DON-AuNPs in the conjugate pad, and saturate the binding sites of the anti-DON antibody in the Ab-AuNP conjugations. Thus, the DON-BSA immobilized at the T1 area cannot capture the saturated anti-DON Ab-AuNP conjugations, and no signal will appear in the T1 area. Similarly, the secondary antibody is immobilized in the T2 area to capture the DON saturated Anti-DON-AuNPs, and shows another color spot. Following the addition of the sample, 40 μL PBS is added to the conjugate pad to wash the channels. After drying, the colorimetric signals in the DON-Chip are imaged using a custom-made portable imaging system. The signal intensities at T1 and T2 areas were calibrated to indicate the DON concentration in the samples.

Detection of DON in spiked samples. To validate the DON-Chip in detecting DON from real world samples, we artificially spiked the corn samples with a DON standard at different concentrations from 0.05 to 1.8 ppm and then conducted the DON detection using the DON-Chip. 0.5 gram of non-infected corn sample was ground and mixed in 1.0 ml of ethyl alcohol solution containing a suitable amount of DON standard. Then the spiked samples were subjected to dissolvent evaporation for 4 h at room temperature. The non-infected corn used in this study was obtained from local market, previously confirmed to contain undetectable levels of DON using the DON-Chip (signal changes in T1 and T2 area are both lower than three times of the standard deviation of blank signal values). The procedures of sample preparation and DON detection were described above.

Interference tests of other mycotoxin and pH. To test the specificity of DON-Chip, equal concentrations (ranging from 1.0-15 ng/mL) of zearalenone (ZEN) were added in the DON standard solutions before the test. The pH interference was tested by adjusting the PBS loading buffers to different pH levels (6.0, 7.2, 8.0 and 9.0).

Portable imaging system. A portable imaging system was developed to facilitate on-site signal reading using the DON-Chip. The system is composed of an XY stage to locate the signal areas, a Z-stage to adjust the focus, and a USB microscope to read the signal. Several custom parts were machined to mount these components on an optical breadboard.

Image capture and signal analysis. The images captured by the portable imaging system were analyzed using the Photoshop CS6 software (Adobe Systems Incorporated, CA, USA). A color image with 1280×1024 pixels that covers the detection areas was captured for signal measurement. The color of images was inverted and then split into three RGB channels and the green channel was used to calculate the signal intensity. Four areas of interest (AOIs) were selected for each channel: T1 area, T2 area, and two blank control area (blank area 1 and blank area 2) around the signal areas. In the histogram of each image, the average value of green channel and the number of pixels was recorded for each AOI. The signal intensities in T1 and T2 areas were calculated using the equation: signal intensity=[average value of AOI— (average value of blank area 1+average value of blank area2)/2]×the number of pixels. The signals from different concentrations (0.0, 1.0, 2.0, 4.0, 8.0, 16, 20 ng/mL) of DON standard were used to generate the calibration curves using four-parameter logistic regression. The signals from four channels (4 replicates, 2 chips) were applied for each detection. We generated three calibration curves from different signal values (T1, T2, T1/T2) and compared their performances. The DON concentration in unknown samples was calculated by fitting the measured signal to the calibration curves.

DON measurement with a commercial ELISA kit. The DON concentrations in the food, feed and feed ingredient samples were measured by a commercial DON ELISA Kit. The signal was read using a Synergy™ H4 Hybrid Multi-Mode Microplate Reader (BioTek, Winooski, Vt., USA) at 450 nm. Before the ELISA test, all the raw samples were initially prepared according to the sample preparation section. The procedures were strictly conducted according to the manufacturer's recommendation.

Statistical analysis. Statistical analyses were performed using GraphPad Prism 8.01 (San Diego, Calif., USA). The Student's t-test was used to compare the DON test data obtained from different conditions or methods. The difference between the two sets of data was considered statistically significant when P value<0.05. One-way ANOVA test was conducted to analyze the difference of total signaling intensity (T1+T2) among the DON standards detections (0.0, 1.0, 2.0, 4.0, 8.0, 16.0, and 20.0 ng/mL).

While the preferred embodiments of the invention have been described above, it will be recognized and understood that various modifications may be made therein, and the appended claims are intended to cover all such modifications which may fall within the spirit and scope of the invention.

TABLE 1
DON measurements in the presence of ZEN. Results were
presented as Means ± SD, n = 4.

| Concentrations¹ | 0 ng/mL ZEN | 1 ng/mL ZEN | 5 ng/mL ZEN | 15 ng/mL ZEN |
|---|---|---|---|---|
| 1 ng/mL DON | 0.87 ± 0.03 | 0.89 ± 0.03 | 0.86 ± 0.06 | 0.87 ± 0.04 |
| 5 ng/mL DON | 5.21 ± 0.27 | 5.14 ± 0.31 | 5.07 ± 0.18 | 5.27 ± 0.26 |
| 15 ng/mL DON | 14.45 ± 0.37 | 14.27 ± 0.47 | 14.47 ± 0.31 | 14.19 ± 0.52 |

¹The Student's t-test was conducted to compare the DON test data under different concentrations of ZEN interference (0, 1, 5 and 15 ng/mL). P < 0.05 was considered a significant difference versus the blank group (0.0 ng/mL ZEN), and presented by

TABLE 2
The influences of pH on DON measurements. Results were
presented as Means ± SD, n = 4.

| Items¹ | pH = 6.0 | pH = 7.2 | pH = 8.0 | pH = 9.0 |
|---|---|---|---|---|
| 1 ng/mL DON | 0.91 ± 0.14 | 0.88 ± 0.07 | 0.90 ± 0.12 | 0.94 ± 0.11 |
| 5 ng/mL DON | 5.31 ± 0.31 | 5.24 ± 0.17 | 5.34 ± 0.24 | 5.52 ± 0.14 |
| 15 ng/mL DON | 14.54 ± 0.41 | 14.23 ± 0.36 | 14.38 ± 0.71 | 14.72 ± 0.57 |

¹The Student's t-test was conducted to compare the DON test data detecting in the different pH values (pH = 6, 7, 8 and 9). P < 0.05 was considered a significant difference versus the control group (pH = 7.2), and presented by

TABLE 3
DON measurements in the spiked corn samples using the DON-Chips.
Results were presented as Means ± SD, n = 4.

| Sample | DON added | Detected | Recovery¹ | CV², intra- |
|---|---|---|---|---|
| No. | (ppm) | concentration (ppm) | (%) | assay (%) |
| 1 | 0.050 | 0.048 ± 0.004 | 96.00 | 8.33 |
| 2 | 0.200 | 0.181 ± 0.012 | 90.05 | 6.62 |
| 3 | 0.600 | 0.625 ± 0.034 | 104.2 | 5.44 |
| 4 | 1.200 | 1.241 ± 0.062 | 103.0 | 5.01 |
| 5 | 1.800 | 1.851 ± 0.096 | 102.8 | 5.19 |

¹Recovery ratio was calculated using the equation: Recovery (%) = (Means/concentration of DON standards) × 100%.
²CV, coefficient of variation of intra-assay was calculated using the equation: CV = (SD/Means) × 100%.

TABLE 4
Results of deoxynivalenol (DON) measurements in
the food, feed and feed ingredient samples. Results
were presented as Mean values ± SEM, n = 4.

| Sample | DON-Chip (ppm)¹ | ELISA (ppm)² | P-value³ |
|---|---|---|---|
| DDGS (1) | 10.24 ± 0.532 | 11.07 ± 0.681 | 0.373 |
| DDGS (2) | 11.64 ± 0.744 | 12.98 ± 0.719 | 0.243 |
| DDGS (3) | 10.93 ± 0.613 | 12.05 ± 0.545 | 0.221 |
| DDGS (4) | 12.06 ± 0.541 | 13.75 ± 0.387 | 0.044 |
| DDGS (5) | 13.58 ± 0.621 | 14.93 ± 0.327 | 0.765 |
| wheat (1) | 0.506 ± 0.032 | 0.472 ± 0.044 | 0.555 |
| wheat (2) | 0.127 ± 0.012 | <0.300 | — |
| wheat (3) | 0.241 ± 0.022 | 0.219 ± 0.016 | 0.449 |
| wheat (4) | 0.843 ± 0.042 | 0.862 ± 0.035 | 0.740 |
| wheat (5) | 0.643 ± 0.057 | 0.687 ± 0.031 | 0.523 |
| Corn (1) | 1.574 ± 0.056 | 1.682 ± 0.082 | 0.319 |
| Corn (2) | 4.524 ± 0.242 | 4.796 ± 0.366 | 0.558 |
| Corn (3) | 3.135 ± 0.457 | 3.149 ± 0.144 | 0.978 |
| Corn (4) | 9.354 ± 0.423 | 10.17 ± 0.315 | 0.172 |
| Corn (5) | 1.504 ± 0.084 | 1.536 ± 0.142 | 0.853 |
| Feed (1) | 2.148 ± 0.091 | 2.311 ± 0.147 | 0.382 |
| Feed (2) | 1.786 ± 0.077 | 1.968 ± 0.372 | 0.649 |
| Feed (3) | 4.327 ± 0.442 | 4.640 ± 0.213 | 0.547 |
| Feed (4) | 1.792 ± 0.041 | 2.044 ± 0.097 | 0.053 |
| Feed (5) | 3.184 ± 0.039 | 3.632 ± 0.508 | 0.510 |
| Unhusked rice | 8.347 ± 0.863 | 9.029 ± 0.711 | 0.564 |

¹A dilution factor of 1000 was applied for distillers dried grains with soluble (DDGS) samples, 500 for corn, feed and unhusked rice samples, and 100 for wheat samples during DON-chip detection.
²A dilution factor of 100 was applied for the ELISA detections.
³The Student's t-test was conducted to compare the DON test data obtained from the different methods. The results obtained from the two detection methods were considered significant different when P value < 0.05.

TABLE 5
Resolvability comparison among the T1,
T2 and T1/T2 based calibration curves.

| | DON concentrations (ng/mL) | | | | |
|---|---|---|---|---|---|
| Items¹ | 0.5-0.6 | 0.6-0.7 | 0.7-0.8 | 0.8-0.9 | 0.9-1.0 |
| Resolvability² | 2.99% | 3.09% | 3.13% | 3.19% | 3.42% |
| (%), T1 | | | | | |
| Resolvability | 9.36% | 9.22% | 8.88% | 8.51% | 8.03% |
| (%), T2 | | | | | |
| Resolvability¹ | 11.17% | 10.98% | 10.53% | 10.34% | 9.93% |
| (%), T1/T2 | | | | | |

¹Calibration equations for T1, T2 and T1/T2 are “Y = {130.62/[1 + (X/2.42)^{1.3}] − 4.39}*10⁴, R² = 0.992”, Y = {130.07 − 113.57/[1 + (X/2.91)^{1.58}]} *10⁴, R2 = 0.995, and Y = 7.92/[1 + (X/0.68)^{1.57}] − 0.016, R² = 0.983, respectively.
²The resolvability on detecting low concentrations (0.5-1.0 ng/mL) of DON was calculated by the equation: Resolvability = |{[Y(n) − Y(n + 0.1)]/Y(n)} | × 100%.

TABLE 6
Unit conversion between ng/mL of deoxynivalenol (DON)
standard and ppm of DON in the raw samples under different
dilution factors (10, 100, 500, and 1000).

| Standard | Raw samples (ppm) under different dilution factors | | | |
|---|---|---|---|---|
| (ng/mL) | 10 | 100 | 500 | 1000 |
| 0.0 | 0 | 0 | 0 | 0 |
| 1.0 | 0.01 | 0.10 | 0.50 | 1.00 |
| 2.0 | 0.02 | 0.20 | 1.00 | 2.00 |
| 4.0 | 0.04 | 0.40 | 2.00 | 4.00 |
| 8.0 | 0.08 | 0.80 | 4.00 | 8.00 |
| 16 | 0.16 | 1.60 | 8.00 | 16.00 |
| 20 | 0.20 | 2.00 | 10.00 | 20.00 |

TABLE 7
Limit of detection (LOD), time per run, detection range and
cost comparisons among the deoxynivalenol (DON) detection
methods of enzyme-linked immunosorbent assay (ELISA), commercial
strip and DON-Chip. The data is from the manual books of commercial
products (commercial products with the best parameter are
chosen for the comparison). The values of LOD and detection
range were uniformly converted, supposing the samples are
under same dilution factor pre-processing.

| Items | ELISA kit | Commercial strip | DON-Chip |
|---|---|---|---|
| LOD, ppb (dilution | 10 | 50 | 4.7 |
| factor, 10) | | | |
| Time per run, minutes | ~120 | ~12 | ~12 |
| Detection range, ppm | 0.02-160 | 0.05-100 | 0.01-20 |
| (dilution factor, 10-1000) | | | |
| Cost, US dollar/duplicate | ~5 | ~4 | 1.94 |
| On-site detection | No | Yes | Yes |

### REFERENCES

(1) Marin, S.; Ramos, A. J.; Cano-Sancho, G.; Sanchis, V. Mycotoxins: occurrence, toxicology, and exposure assessment. Food Chem Toxicol 2013, 60, 218-37.
(2) Terciolo, C.; Maresca, M.; Pinton, P. Oswald, I. P., Review article: Role of satiety hormones in anorexia induction by Trichothecene mycotoxins. Food Chem Toxicol 2018, 121, 701-14.
(3) Clifford, L. J.; Jia, Q.; Pestka, J. J. An improved method for the purification of the trichothecene deoxynivalenol (vomitoxin) from Fusarium graminearum culture. J Agric Food Chem 2003, 51, 521-3.
(4) Sobrova, P.; Adam, V.; Vasatkova, A.; Beklova, M.; Zeman, L.; Kizek, R. Deoxynivalenol and its toxicity. Interdiscip Toxicol 2010, 3, 94-9.
(5) Gauthier, T.; Wache, Y.; Laffitte, J.; Taranu, I.; Saeedikouzehkonani, N.; Mori, Y.; Oswald, I. P. Deoxynivalenol impairs the immune functions of neutrophils. Mol Nutr Food Res 2013, 57, 1026-36.
(6) Islam, M. R.; Roh, Y. S.; Kim, J.; Lim, C. W.; Kim, B., Differential immune modulation by deoxynivalenol (vomitoxin) in mice. Toxicol Lett 2013, 221, 152-63.
(7) Lessard, M.; Savard, C.; Deschene, K.; Lauzon, K.; Pinilla, V. A.; Gagnon, C. A.; Lapointe, J.; Guay, F.; Chorfi, Y. Impact of deoxynivalenol (DON) contaminated feed on intestinal integrity and immune response in swine. Food Chem Toxicol 2015, 80, 7-16.
(8) Clark, E. S.; Flannery, B. M.; Gardner, E. M.; Pestka, J. J. High Sensitivity of Aged Mice to Deoxynivalenol (Vomitoxin)-Induced Anorexia Corresponds to Elevated Proinflammatory Cytokine and Satiety Hormone Responses. Toxins (Basel) 2015, 7, 4199-215.
(9) Trenholm, H. L.; Cochrane, W. P.; Cohen, H.; Elliot, J. I.; Farnworth, E. R.; Friend,
D. W.; Hamilton, R. M.; Standish, J. F.; Thompson, B. K. Survey of vomitoxin contamination of 1980 Ontario white winter wheat crop: results of survey and feeding trials. J Assoc Off Anal Chem 1983, 66, 92-7.
(10) Maresca, M.; Yahi, N.; Younes-Sakr, L.; Boyron, M.; Caporiccio, B.; Fantini, J. Both direct and indirect effects account for the pro-inflammatory activity of enteropathogenic mycotoxins on the human intestinal epithelium: stimulation of interleukin-8 secretion, potentiation of interleukin-1beta effect and increase in the transepithelial passage of commensal bacteria. Toxicol Appl Pharmacol 2008, 228, 84-92.
(11) Bonnet, M. S.; Roux, J.; Mounien, L.; Dallaporta, M.; Troadec, J. D. Advances in deoxynivalenol toxicity mechanisms: the brain as a target. Toxins (Basel) 2012, 4, 1120-38.
(12) Bae, H.; Gray, J. S.; Li, M.; Vines, L.; Kim, J.; Pestka, J. J. Hematopoietic cell kinase associates with the 40S ribosomal subunit and mediates the ribotoxic stress response to deoxynivalenol in mononuclear phagocytes. Toxicol Sci 2010, 115, 444-52.
(13) USFDA (United States Food and Drugs Administration), undated. FDA Regulatory Guidance for Toxins and Contaminants. http://www.ngfa.org/toxinsPDF-1.pdf.
(14) Guerre, P. Worldwide Mycotoxins Exposure in Pig and Poultry Feed Formulations. Toxins 2016, 8, 350-74.
(15) Ran, R.; Wang, C. H.; Han, Z.; Wu, A. B.; Zhang, D. B.; Shi, J. X. Determination of deoxynivalenol (DON) and its derivatives: Current status of analytical methods. Food Control 2013, 34, 138-48.
(16) Chen, A.; Yang, S. Replacing antibodies with aptamers in lateral flow immunoassay. Biosens Bioelectron 2015, 71, 230-42.
(17) Kolosova, A. Y.; De Saeger, S.; Sibanda, L.; Verheijen, R.; Van Peteghem, C. Development of a colloidal gold-based lateral-flow immunoassay for the rapid simultaneous detection of zearalenone and deoxynivalenol. Anal Bioanal Chem 2007, 389, 2103-7.
(18) Kolosova, A. Y.; Sibanda, L.; Dumoulin, F.; Lewis, J.; Duveiller, E.; Van Peteghem, C.; De Saeger, S. Lateral-flow colloidal gold-based immunoassay for the rapid detection of deoxynivalenol with two indicator ranges. Anal Chim Acta 2008, 616, 235-44.
(19) Urusov, A. E.; Gubaidullina, M. K.; Petrakova, A. V.; Zherdev, A. V.; Dzantiev, B. B. A new kind of highly sensitive competitive lateral flow immunoassay displaying direct analyte-signal dependence. Application to the determination of the mycotoxin deoxynivalenol. Mikrochim Acta 2017, 185, 29.
(20) Mallmann, A. O.; Oliveira, M. S.; Dilkin, P.; Vidal, J. K.; Meinerz, G. R.; Almeida, C. A. A.; Mallmann, C. A.. Assessment of mycotoxin contamination in maize and wheat stored in silos using two sampling processes. World Mycotoxin J 2018, 11, 531-538.
(21) Henares, T. G.; Mizutani, F.; Hisamoto, H. Current development in microfluidic immunosensing chip. Anal Chim Acta 2008, 611, 17-30.
(22) Gong, M. M.; Sinton, D. Turning the Page: Advancing Paper-Based Microfluidics for Broad Diagnostic Application. Chem Rev 2017, 117, 8447-80.
(23) Ren, K. N.; Chen, Y.; Wu, H. K. New materials for microfluidics in biology. Curr Opin Biotechnol 2014, 25, 78-85.
(24) Casto, L. D.; Schuster, J. A.; Neice, C. D.; Baker, C. A. Characterization of low adsorption filter membranes for electrophoresis and electrokinetic sample manipulations in microfluidic paper-based analytical devices. Anal Metho 2018, 10, 3616-23.
(25) Fu, L. M.; Wang, Y. N. Detection methods and applications of microfluidic paper-based analytical devices. Trends Analyt Chem 2018, 107, 196-211.
(26) Wang, T. F.; Xu, G. N.; Wu, W. Y.; Wang, X. Y.; Chen, X. L.; Zhou, S. M.; You, F. A novel combination of quick response code and microfluidic paper-based analytical devices for rapid and quantitative detection. Biomed Microdevices 2018, 20.
(27) Pleadin, J.; Sokolovic, M.; Persi, N.; Zadravec, M.; Jaki, V.; Vulic, A. Contamination of maize with deoxynivalenol and zearalenone in Croatia. Food Control 2012, 28, 94-8.

1. A method for detecting levels of deoxynivalenol in a sample comprising:
providing an assay support comprising a sample loading area connected by a channel to a first test area and a second test area;
said sample loading area comprising a quantity of anti-deoxynivalenol compound binding antibodies;
said first test area comprising a quantity of deoxynivalenol compound bound to a carrier;
said second test area comprising a quantity of anti-deoxynivalenol compound binding antibodies binding reagent;
wherein said sample flows from the sample loading area along the channel to the first test area and then along the channel to the second test area;
loading a sample to be tested for a deoxynivalenol compound onto the sample loading area such that contents of the sample interact with the quantity of anti-deoxynivalenol compound binding antibodies, a portion of said quantity of anti-deoxynivalenol compound binding antibodies forming anti-deoxynivalenol compound binding antibody:deoxynivalenol compound complexes, a remaining portion of the quantity of anti-deoxynivalenol compound binding antibodies remaining unbound anti-deoxynivalenol binding antibodies;
said sample comprising the anti-deoxynivalenol compound binding antibody:deoxynivalenol complexes and the unbound anti-deoxynivalenol compound binding antibodies flowing along the channel to the first test area, said unbound anti-deoxynivalenol compound binding antibodies binding to the quantity of deoxynivalenol compound bound to a carrier and being retained in the first test area;
said sample comprising the anti-deoxynivalenol compound binding antibody:deoxynivalenol compound complexes continuing to flow along the channel to the second test area, said anti-deoxynivalenol compound binding antibody:deoxynivalenol compound complexes binding to the quantity of anti-deoxynivalenol compound binding antibodies binding reagent and being retained in the second test area; and
measuring the deoxynivalenol compound level in the sample by detecting the anti-deoxynivalenol compound binding antibodies at the first test area and/or detecting the anti-deoxynivalenol compound binding antibodies at the second testing area.

2. The method according to claim 1 wherein the anti-deoxynivalenol compound antibodies comprise a detectable label.

3. The method according to claim 2 wherein the anti-deoxynivalenol compound antibodies are labeled with gold nanoparticles.

4. The method according to claim 1 wherein the assay support is a paper-based microfluidic chip.

5. The method according to claim 4 wherein the paper-based microfluidic chip is composed of nitrocellulose paper.

6. The method according to claim 1 wherein the sample is a food sample or a feed sample.

7. The method according to claim 1 wherein the deoxynivalenol compound level is determined by detecting the anti-deoxynivalenol compound binding antibodies at the first test area and detecting the anti-deoxynivalenol compound binding antibodies at the second testing area.

8. The method according to claim 7 wherein the deoxynivalenol compound level is determined by the ratio of anti-deoxynivalenol compound binding antibodies at the first test area to the anti-deoxynivalenol compound binding antibodies at the second testing area.

9. The method according to claim 1 wherein the channel has curved corners.

10. The method according to claim 1 wherein the first testing area and the second testing area are separated by a separation zone.

11. The method according to claim 1 wherein the deoxynivalenol compound is selected from the group consisting of deoxynivalenol, 3-acetyl deoxynivalenol and 15-acetyl deoxynivalenol.

12. The method according to claim 1 wherein the deoxynivalenol compound bound to a carrier is selected from the group consisting of deoxynivalenol, 3-acetyl deoxynivalenol and 15-acetyl deoxynivalenol.

13. The method according to claim 1 wherein the assay support further comprises an absorbent zone and the sample flows along the channel from the second test area to the absorbent zone.

14. The method according to claim 1 wherein the anti-deoxynivalenol binding antibodies binding reagents are secondary antibodies.

15. A method for manufacturing a device for detecting deoxynivalenol in a sample comprising:
providing an assay support comprising a sample loading area connected by a channel to a first test area and a second test area;
depositing a quantity of anti-deoxynivalenol compound binding antibodies at the sample loading area;
depositing a quantity of anti-deoxynivalenol compound bound to a carrier at the first test area; and
depositing a quantity of anti-deoxynivalenol compound binding antibody binding reagent at the second testing area.

16. The method according to claim 15 wherein the anti-deoxynivalenol compound antibodies comprise a detectable label.

17. The method according to claim 16 wherein the anti-deoxynivalenol compound antibodies are labeled with gold nanoparticles.

18. The method according to claim 15 wherein the assay support is a paper-based microfluidic chip.

19. The method according to claim 18 wherein the paper-based microfluidic chip is composed of nitrocellulose paper.

20. The method according to claim 15 wherein the channel has curved corners.

21. The method according to claim 15 wherein the first testing area and the second testing area are separated by a separation zone.
